(19) United States
(12) Patent Application Publication
(10) Pub. No.: US 2010/0331878 A1
(43) Pub. Date: Dec. 30, 2010

(54) METHOD AND DEVICE FOR REMOVING A BALLOON FROM A BODY CAVITY

(75) Inventor: Timo Kleinwachter, Munsingen (DE)

Correspondence Address: Gerald E. Hespos, Esq.;CASELLA & HESPOS LLP, Suite 1703, 274 Madison Avenue, New York, NY, 10016, US

(73) Assignee: CARL ZEISS SURGICAL GMBH, Oberkochen (DE)

(21) Appl. No.: 12/491,603

(22) Filed: Jun. 25, 2009

(51) Int. Cl. A61B 17/28

(52) U.S. Cl. 606/205

(57) ABSTRACT

A balloon removing device is provided for removing a balloon from a body cavity through an access opening of said body cavity, in particular for removing a balloon of a radiation catheter from a body cavity left by surgical removal of cancerous or other tissue. The balloon removing device has an unstretchable elongated tool extending along a longitudinal axis and having a distal end with a distal opening, a proximal end with a proximal opening and a lumen extending from the distal opening along the longitudinal axis to the proximal opening. The dimensions of the distal opening and the lumen of the elongated tool allow the balloon to be accommodated therein in the deflated state, or the dimensions of the distal opening, the proximal opening and the lumen of the elongated tool are adapted to allow the balloon to be pulled through the lumen in the deflated state.

### BACKGROUND OF THE INVENTION

1. Field of the Invention

The present invention relates to removing a balloon from a body cavity of a patient, and in particular to a method and a device for removing a balloon of a radiation catheter for locally treating body tissue, through a natural or artificial access opening of the body cavity.

2. Description of the Related Art

Treatment of cancer may include removal of a tumour by a surgical intervention. After the tumour has been removed, radiation is often used to further treat a cancer in order to insure that cancer cells that remain in the tumour area are destroyed. One method of radiating remaining tumour cells is to apply an external radiation source to irradiate the location where the tumour has been removed transdermally. However, such radiation treatments usually expose a relatively large body area to the radiation.

In order to more locally irradiate the target areas for a radiation treatment radiation catheters have been developed which can be introduced into the body cavity created by the removal of the tumour. Such catheters are introduced into the body cavity through an incision which is kept as small as possible. Therefore, often inflatable balloons are used which will be inflated after introducing them into the body cavity. Moreover, the inflated balloon conforms the tissue lining of the cavity about the balloon surface to achieve a more uniform and controlled application of radiation. Such catheters are, for example, disclosed in US 2004/0087827 A1, US 2008/228025 A1, WO 2007/145965 A2 and US 2006/0205992 A1.

The radiation catheters usually remain in the body cavity during the treatment for a period of about two days to several weeks. As it is, in particular, described in US 2006/0205992 A1 the catheter shaft may be folded or coiled and placed under the patient's skin during the treatment. When the treatment is over the incision is reopened and the shaft which has been located under the skin is unfolded or uncoiled. Then the balloon is deflated and retrieved from the body cavity in the deflated state.

Insertion and retrieval of an inflatable balloon not having a shaft such as balloons for treating incontinence can be done by using an insertion and retrieval system as it is, for example, disclosed in WO 03/075791 A2. Such a system comprises a capturing member and a needle sufficient to pierce the balloon. For retrieval the balloon is pierced by the needle and the contents of the balloon is sucked out. Then, the deflated balloon is retrieved out of the body cavity. The capturing device is embodied as a magnet that attracts a special element of the balloon, so that, during retrieval, the balloon is held at the distal end of the capturing device.

However, retrieval of a deflated balloon from a natural or artificial body cavity can cause pain to the patient. Typically, the balloon crumples during deflation to form wrinkles which may have diameters which are larger than the diameter of the access opening to the body cavity. In this case, the wrinkles stretch the access opening causing pain to the patient.

Hence, there is a need in the art for an improved method and an improved device for removing the balloon of a catheter from a body cavity though an access opening.

### SUMMARY OF THE INVENTION

The invention provides a balloon removing device, in particular for removing the balloon of a catheter, like the balloon of a radiation catheter, from a body cavity through an access opening of said body cavity. The body cavity may natural or may be left by surgical removal of cancerous or other tissue. In the context of the present invention the term “balloon” shall represent an inflatable device which can be inflated by introducing a fluid, i.e. a gas or a liquid, into a volume of the device.

The inventive balloon removing device comprises an instretchable elongated tool extending along a longitudinal axis and having a distal end with a distal opening, a proximal end with a proximal opening and a lumen extending from the distal opening along a longitudinal axis to the proximal opening. The dimensions of said distal opening and said lumen of the elongated tool are adapted to allow the balloon to be accommodated therein in the deflated state, or the dimensions of said distal opening, said proximal opening and said lumen of the elongated tool are adapted to allow the balloon to be pulled trough the lumen in the deflated state.

According to another aspect, the invention provides a method of removing a balloon from a body cavity through an access opening of said body cavity, where the body cavity may be a natural body cavity, such as the bladder, or an artificial body cavity resulting from the removal of body tissue. In particular, the invention provides a method of removing a balloon of a radiation catheter from a body cavity left by surgical removal of cancerous or other tissue. The method comprises the steps of
inserting an instretchable elongated tool extending along a longitudinal axis and having a distal end with a distal opening, a proximal end with a proximal opening, and a lumen extending from the distal opening to the proximal opening, through the access opening into the body cavity
placing the balloon in the lumen of the elongated tool and retrieving the balloon placed in the lumen together with the elongated tool from the body cavity, or retrieving the balloon from the body cavity through the lumen of the elongated tool.

The dimension of the lumen of the elongated tool defines the size of the opening available for retrieving the balloon from the body cavity. Since the instretchable elongated tool can not be stretched by the balloon, wrinkles of the balloon are tilted when the balloon is pulled into or through the elongated tool, thereby preventing the balloon from having a larger diameter than the access opening. Hence, stretching of the access opening can be reliably avoided by using the elongated tool.

The elongated tool may have an inner surface which surrounds the lumen and which has a low friction, thereby facilitating movement of the deflated balloon into or through the lumen of the elongated tool. The low friction inner surface may, for example, be formed by a low friction coating applied to the material of the elongated tool, such as, e.g., a polytetrafluoroethylene coating (PTFE).

The distal opening of the elongated tool may have a larger cross section than the lumen. A tapering wall section then extends from the distal opening in axial direction the tapering wall section having a cross section which scales down from the cross section of the distal opening to the cross section of the lumen. Hence, the distal end of the elongated tool forms a funnel shaped entrance to the lumen which assists in tilting the wrinkles.

The elongated tool of the inventive device may, for example, be implemented as a hollow tube.

In a special implementation of the inventive device the elongated tool is formed from two separate pieces which are movable relative to each other in a direction perpendicular to the longitudinal axis of the tool. A handling element which comprises two handling pieces is present in this implementation. Each piece of the elongated tool is attached or attachable to a different one of the handling pieces. The handling pieces are movably connected to each other so as to allow to impart a movement of the attached pieces of the elongated tool in a direction perpendicular to the longitudinal axis of the elongated tool by moving the handling pieces relative to each other. This simplifies removal of a balloon which has been retrieved from the body cavity while being placed in the lumen from the lumen by just moving the two pieces of the elongated tool away from each other.

The handling element of such an implementation may be a tweezers-like, a pliers-like or a scissors-like element wherein the handling pieces are in form of levers each having a manipulation section or end for manipulating the handling element and an attaching section or end to which one of said pieces of the elongated tool is attached or attachable and wherein the levers are joined together, so as to allow for moving the attaching section or end. The levers may be, in particular, pivotally joined together. Said pieces of the elongated tool may, in particular, extend substantially perpendicular to the levers of the handling element.

For preventing the balloon from slipping out of the lumen of the elongated tool the tool may comprise a holding element which may, e.g., comprise one or more resilient tongues projecting from a wall of the elongated tool towards the proximal opening into the lumen.

Often, a catheter shaft is still connected to the balloon when the balloon is retrieved from the body cavity. This catheter shaft can be used to pull the balloon through the lumen of the elongated member or to move the balloon into the lumen and together with the elongated member out of the body cavity. However, it is conceivable that there is no catheter shaft connected to the balloon when the balloon is to be retrieved from the body cavity. For this case the balloon catheter removing device may comprise a gripping element which allows for gripping the balloon and which can be moved back and forth through the lumen of the elongated tool. The gripping element may, e.g. have a hook-like or claw-like structure at it distal end. By using such a gripping element the inventive method can further comprise the step of gripping the balloon with the gripping element which can be moved back and forth through the lumen of that elongated tool and, after gripping the balloon with the gripping element, retrieving the balloon from the body cavity.

According to another aspect of the invention, a balloon removing hemostat is provided having a first lever, a second lever and a tube having first semi-tubular piece and a second semi-tubular piece which are made of an instretchable material and which can abut each other so as to form said tube. Each lever has a grip end for handling the hemostat and a tool end to which one of the semi-tubular pieces are attached or attachable. The levers are pivotally joined at a location between the grip ends and the tool ends so as to allow a back and forth movement of the semi-tubular pieces from a position where the semi-tubular pieces are separate from each other into a position where the semi-tubular pieces abut each other by handling the grip ends.

If a catheter shaft is still connected to the balloon when the balloon is to be retrieved from the body cavity the semi-tubular pieces of the balloon catheter removing hemostat can be closed around the shaft and then moved along the shaft into the body cavity. The balloon can then be retrieved by pulling it out of the body cavity through the lumen of the tube or by placing the balloon into the lumen and pulling the balloon together with the tube out of the body cavity.

The balloon removing hemostat may comprise a rod having a proximal end, a distal end, and a hook or claw located at its distal end. The rod is slidably inserted or insertable into the lumen of the tube so that the hook or claw can be moved out of the distal end of the tube so as to fasten the balloon to the hook or claw and back into or though the tube when the balloon is fastened to the hook or claw. This allows for retrieving balloons to which no catheter shaft is connected from a body cavity.

According to a further aspect of the present invention, a balloon catheter removing tube is provided which is made of an instretchable material and which is insertable into a body cavity through an access opening of the body cavity. The tube has an axial direction, radial direction, a distal end with a distal opening, a proximal end with a proximal opening and a lumen extending from the distal opening in axial direction of the tube to the proximal opening. The dimensions of the distal opening and the lumen are adapted to allow a balloon to be accommodated in the lumen in the deflated state or the dimension of the distal opening, the proximal opening and the lumen are adapted to allow the balloon to be pulled through the lumen in the deflated state.

The balloon removing tube may comprise a rod having a proximal end, a distal end, and a hook or claw located at its distal end, the rod being slidably inserted or insertable into the lumen of the tube so that the hook or claw can be moved out of the distal end of the tube so as to fasten the balloon to the hook or claw and back into or through the tube when the balloon is fastened to the hook or claw.

### BRIEF DESCRIPTION OF THE DRAWINGS

FIG. 1 shows a first embodiment of the inventive balloon removing device in the perspective view.

FIG. 2 shows the balloon removing device of FIG. 1 in a top view.

FIG. 3 shows the balloon removing device of FIG. 1 in a side view.

FIG. 4 shows a first step of an embodiment of the inventive method of removing a balloon from a body cavity.

FIG. 5 shows a second step of the embodiment of the method of removing a balloon from a body cavity.

FIG. 6 shows a modification of the embodiment of the inventive method using a modified balloon removing device.

FIG. 7 shows a modification of the embodiment of the balloon removing device shown in FIGS. 1 to 3.

FIG. 8 shows a second embodiment of the inventive balloon removing device in a perspective view.

FIG. 9 shows the second embodiment in a sectional view along its longitudinal axis.

FIG. 10 shows a modification of the second embodiment of the inventive balloon removing device.

FIG. 11 shows a gripping element of the inventive balloon removing device.

### DETAILED DESCRIPTION OF THE PREFERRED EMBODIMENTS

In the following details description of the preferred embodiments, reference is made to the accompanying drawings, which from a part hereof, and within which are shown by way of illustration specific embodiments by which the invention may be practiced. It is to be understood that other embodiments may be utilized and structural changes may be made without departing from the scope of the invention.

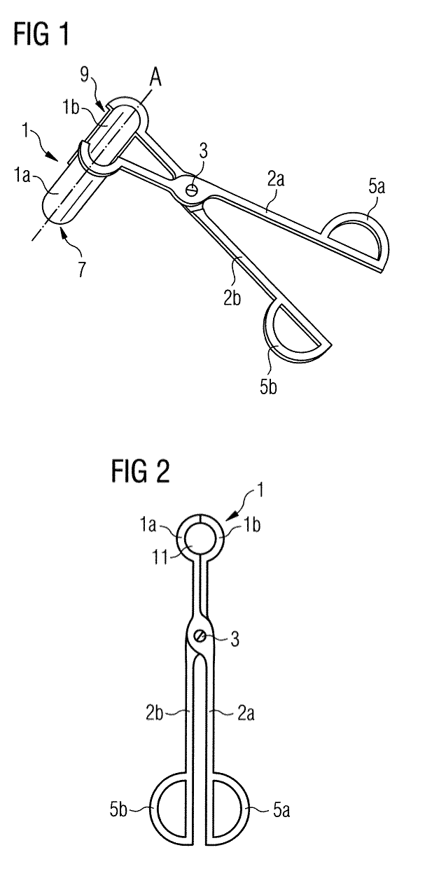

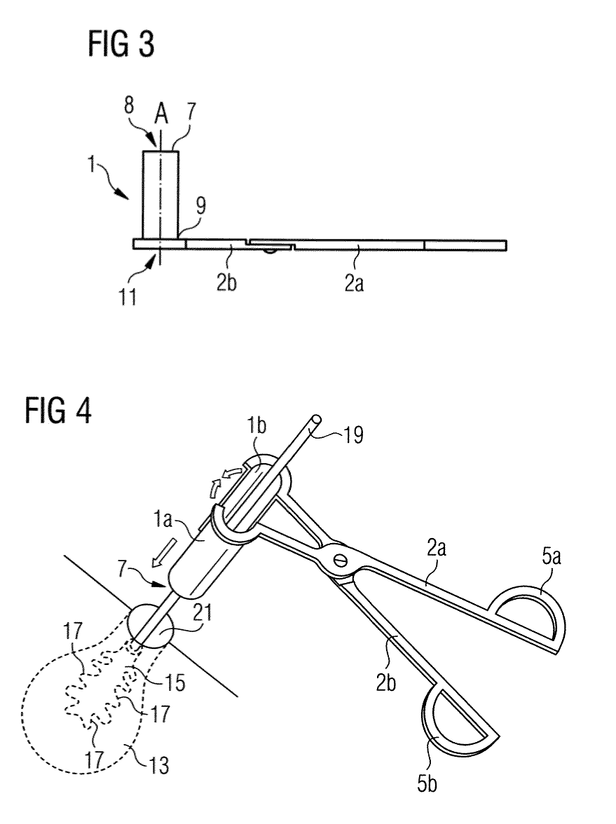

A first embodiment of the inventive balloon removing device for removing a balloon from a body cavity trough an access opening will be described with reference to FIGS. 1 to 3. FIG. 1 shows a balloon removing hemostat as first embodiment of the inventive balloon removing device in a perspective view. FIG. 2 shows the balloon removing hemostat in a top view and FIG. 3 shows the balloon removing hemostat in a side view.

The balloon removing hemostat includes a pair of levers 2a, 2b which are pivotally joined by a hinge 3. The levers 2a, 2b together form a scissors-like instrument with one end of each lever 2a, 2b forming a handle 5a, 5b representing a grip end of the balloon removing hemostat for handling the hemostat. At the other ends of the levers 2a, 2b semi-tubular pieces 1a, 1b made from an instretchable material are attached which extend substantially perpendicular to the extension of the levers 2a, 2b. The semi-tubular pieces 1a, 1b, which may, e.g., be made of metal or an instretchable plastic material, are attached such to the levers 2a, 2b that they can be brought into contact with each other at their respective open sides so as to form a tube 1 when they abut each other. The tube 1 then forms an instretchable elongated tool extending along a longitudinal axis A and having a proximal end 9 connected to the levers 2a, 2b and a distal end 7 which can be inserted into an access opening of a body cavity. In the state where the semi-tubular pieces 1, 1b abut each other to form the tube 1 the inside of the tube 1 forms a lumen with a distal opening 8 at the distal end 7 and a proximal opening 11 at the proximal end 9. While FIG. 1 shows the balloon removing hemostat in an open state of the tube 1, i.e. with the semi-tubular pieces 1a, 1b moved away from each other, FIGS. 2 and 3 show the balloon removing hemostat in the closed state of the tube 1. Note that although the semi-tubular pieces 1a, 1b are attached to the ends of the levers 2a, 2b in the present embodiment they may as well be formed as integral parts of the levers.

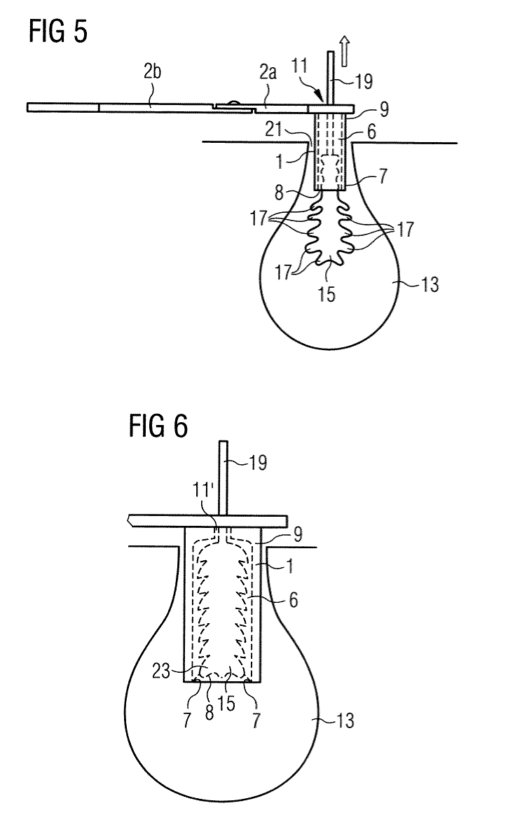

An embodiment of the inventive method of removing a balloon from a body cavity will now be described with respect to FIGS. 4 and 5. In the shown embodiment of the inventive method the balloon removing hemostat which has been described with respect to FIGS. 1 to 3 is used.

The balloon to be removed from a body cavity 13 is, in the present embodiment of the inventive method, part of a radiation catheter which is typically implanted into a body cavity for radiation treatment of remaining tumour cells after surgical removal of a tumour. The radiation catheter comprises a balloon 15 and a shaft 19 connected to the balloon 15. The shaft 19 is typically folded or coiled and placed under the patient's skin during the treatment period of several hours to several weeks. When the balloon catheter is to be removed an access opening 21 is created or reopened and the shaft 19 is unfolded or unrolled so as to project out of the access opening 21. Then, the balloon 15 is deflated whereby it forms wrinkles 17 which may lead to a balloon diameter perpendicular to the shaft axis which is larger than the diameter of the access opening 21. Removing the balloon in this wrinkled state without using the inventive tool could lead to stretching of the access opening which would cause pain to the patient.

Therefore, in a first step of the inventive method, the balloon removing hemostat is used to place the semi-tubular pieces 1a, 1b laterally around the catheter shaft 19. Then the semi-tubular pieces 1a, 1b are brought together so as to about each other and to form the tube 1, and the distal end 7 of the tube 1 is inserted into the body cavity 13 through the access opening 21 to arrive at the state shown in FIG. 5.

After the distal end 7 of the tube 1 has been introduced into the body cavity 13 through the access opening 21 the balloon 15 is pulled through the lumen 6 of the tube 1 by means of the catheter shaft 19. When entering the lumen 6 through the distal opening 8 of the tube 1 the wrinkles 17 will be folded towards the centre of the balloon 15 so as to compress the balloon into the lumen 6 as the tube 1 can not be stretched by the deflated balloon. Hence, the tube 1 prevents access opening 21 from being stretched when the balloon 15 is retrieved.

According to the inventive method, the balloon 15 can be pulled through the lumen 6 of the tube 1, i.e. so that it is completely removed out of the lumen 6 through the proximal opening 11 before the tube 1 is pulled out of the access opening 21 again. Another possibility is to pull the balloon 15 into the lumen 6 and leaving the balloon there. Then the tube 1 is pulled out of the access opening 21 together with the balloon 15 accommodated in the interior of the tube 1. After the tube has been retracted from the body cavity 13, the balloon 15 can easily be removed from the interior of the tube 1, i.e. from the lumen, by moving the semi-tubular pieces 1 a, 1 b away from each other.

Of course the diameter of the lumen 6 of the elongated tool, i.e. the inner diameter of the tube 1 in the present embodiment of the balloon removing device, is adapted so as to be able to allow the balloon 15 to be pulled through or to be accommodated in the lumen 6. This may mean that elongated tools like the tube 1 having different diameters need to be used for balloons of different sizes. However, it is desirable to use an elongated tool having the smallest diameter possible for removing a balloon having a given size since this allows for using an as small as possible access opening 21.

A modification of the balloon removing hemostat as it has been described with respect to FIGS. 1 to 3 is shown in FIG. 6. While in the balloon removing hemostat of FIGS. 1 to 3 the distal opening 8 and the proximal opening 11 are of the same diameter so as to allow to pull the balloon through the lumen 6 the diameter of the proximal opening 11′ of the modification shown in FIG. 6 is smaller than the diameter of the distal opening 8. In fact, the diameter of the proximal opening 11 can be so small that it is just large enough to allow feeding the catheter shaft 19 there trough. The size of the lumen 6 of the tube 1, i.e. its diameter and length, is chosen such that the balloon 15 can be fully accommodated in it. For retrieval of the balloon 15 from body cavity 13 the semi-tubular pieces are closed around the shaft 19, as it has been described with respect to FIG. 4, and the tube 1 is inserted into the body cavity 13. Then, the balloon 15 is pulled into the lumen 6 by pulling at the shaft 19. The tube 1 with the balloon 15 accommodated in the lumen 6 is shown in FIG. 6. Then, the tube 1 is moved out of the body cavity 13 together with the balloon 15 accommodated therein.

In order to prevent the balloon 15 from slipping out of the lumen 6 at least one resilient tongue 23 may be present which projects radially inwards from the rim of the distal opening 8 and backwards, i.e. towards the proximal end 9 of the tube 1. This construction allows the balloon 15 to be easily pulled into the lumen 6 while, on the other hand, providing a considerable resistance against the balloon 15 slipping out of the lumen 6 through the distal opening 8.

Although the described tongues 23 are helpful for preventing the balloon 17 from slipping out of the lumen 6 they are not mandatory in the context of the invention since the balloon can also be held in the lumen 6 by pulling at the catheter shaft 19. On the other hand, the tongues described with respect to the modification shown in FIG. 6 can also be present in the embodiment shown in FIGS. 1 to 3 or in any of the other embodiments described below. Moreover, they do not need to be located at the rim of the distal opening 7. Alternatively or additionally, tongues may be present anywhere inside the lumen 6, preferably closer to the distal end 7 of the tube 1 than to the proximal end 9.

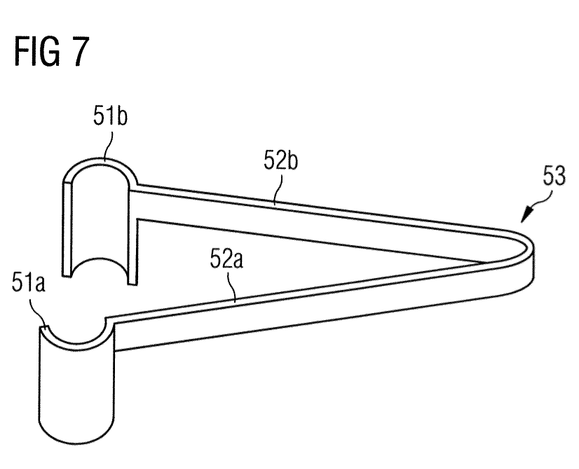

A further modification of the first embodiment of the inventive balloon removing device is shown FIG. 7. In the modification, the inventive balloon removing device is embodied tweezers-like rather than scissors-like. The tweezers-like balloon removing device comprises a single piece of a flat elongated elastic material, like metal or a suitable plastic material, with a bent portion 53. Two levers 52a, 52b having the same length extend from the bent portion 53. Semi-tubular pieces 51a, 51b with open sides are integrally formed at the ends of the levers 52a, 52b. By moving the levers 52a, 52b towards each other the semi-tubular pieces 51a, 51b can be brought into contact with each other at their respective open sides so as to form a tube when they abut each other. The tube then forms an instretchable elongated tool extending along a longitudinal axis. The semi-tubular pieces 51a, 51b and the tube correspond to those described with respect to FIGS. 1 to 6.

Note, that Instead of being integrally formed with the levers 52a, 52b the semi-tubular pieces 51a, 51 may be attached or attachable to the ends of the levers 52a, 52b. Furthermore, instead being made of single piece of a flat elongated elastic material the two levers may be made from separate elongated pieces which are joined together at their ends. In this case the elongated pieces do not need to be made of an elastic material since the elongated pieces can be hinged together with an elastic element like, for example, a spring being located between the elongated pieces so as to force them apart. However, if the two levers are made of an elastic material a fixed joint like, for example, a glue joint or weld joint, are sufficient.

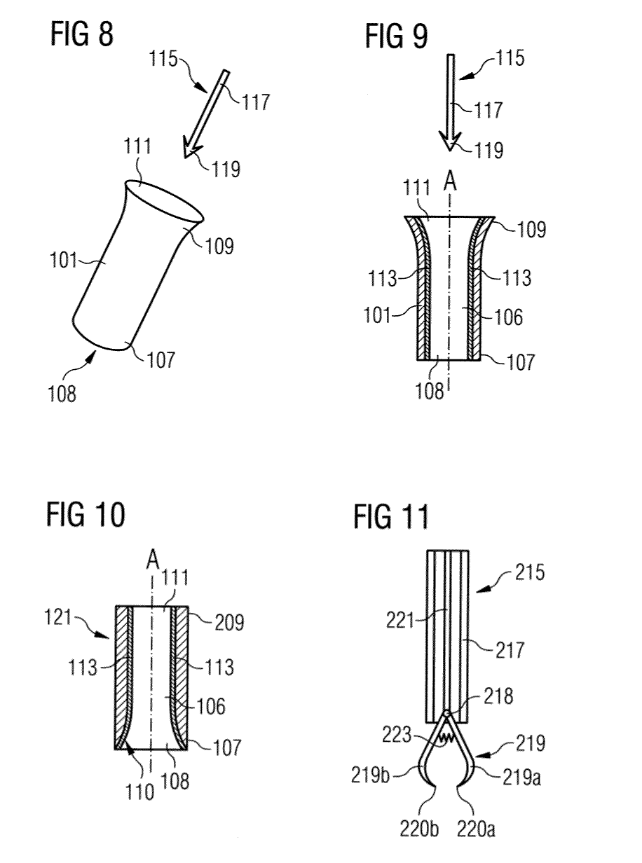

A second embodiment of the inventive balloon removing device will now be described with respect to FIGS. 8 and 9. The balloon removing device according to the second embodiment comprises a tube 101 as an elongated tool. The tube 101 comprises a distal end 107 with a distal opening 108, a proximal end 109 with a proximal opening 111 and a lumen 106 extending from the distal opening 108 to the proximal opening 111 along a longitudinal axis A of the tube 101. The tube 101 is made of an instretchable material such as, e.g., metal or an instretchable plastic material. While the tube 101 is shown in a prospective view in FIG. 7 FIG. 8 shows a sectional view along the longitudinal axis A.

As can be seen in FIG. 9, the inside of the tube 101 is covered by a PTFE-coating to reduce friction of the tube inside surface. Although PTFE is described as material of the friction reducing coating 113, other solid lubricants can be used as well. Furthermore, instead of applying a solid lubricant in form of a coating to the inside of the tube 101 to form a surface having low friction the tube itself can be made of a material having low friction. For example, the whole tube 101 could, in principle, be made of PTFE.

When using the tube 101 shown in FIGS. 8 and 9 instead of the balloon removing hemostat described with respect to FIGS. 1 to 3 in the inventive method of removing the balloon of a balloon catheter from a body cavity the method is modified in that the tube is slipped over the proximal end of the catheter shaft 19 and then inserted into the access opening of the body cavity 13. In order to prevent the tube 101 from slipping to far into the body cavity 13 through the access opening 21 the proximal end 109 of the tube may have a larger diameter than the rest of the tube 101, as it is shown in FIGS. 8 and 9.

In case the shaft 19 of the catheter comprises elements of a diameter larger than the diameter of the lumen 106 of the tube 101 such parts of the catheter shaft 19 can be cut. The catheter shaft can also be cut if it is very long. However, a piece of the shaft should remain which is long enough to pull the balloon into or through the lumen 106 of the tube 1.

Note that although not shown in FIGS. 8 and 9 at least one tongue like those described with respect to FIG. 6 can be provided at the rim of the distal opening 108 and/or in the lumen of the tube 101.

A modification of the second embodiment of the inventive balloon removing device is shown in FIG. 10 in a sectional view along the longitudinal axis A of the tube 121. The main difference of the tube 201 shown in FIG. 10 to the tube 101 shown in FIGS. 8 and 9 is the fact that the distal opening 108 has a larger cross section than the lumen 106 and that the tube 121 comprises a tapering wall section 110. The cross section of the tapering wall section 110 scales down from the cross section of the distal opening 108 to the cross section of the lumen 106. Hence, a funnel shaped tube section extends from the distal opening in the direction towards the proximal opening 111 which simplifies moving the balloon into the lumen 106.

Although the modification shown in FIG. 10 does not have increasing tube dimensions at the proximal end 109 this modification can as well have a proximal end as it is shown in FIGS. 8 and 9. In addition, the tapering wall section 110 is not only applicable to the embodiment shown in FIG. 10 but also to all other embodiments, and in particular to those shown in FIGS. 1 to 3. In addition, like the embodiment shown in FIG. 6, the proximal end opening 111 of the embodiment shown in FIGS. 8 and 9 as well as of its modification shown in FIG. 10 can have a smaller diameter than the diameter of the lumen. In particular the diameter of the proximal opening may be chosen such that it is just large enough to allow the shaft of a balloon catheter to pass through the opening.

Up to now, retrieval of balloons from which a catheter shaft 19 extends by use of an inventive balloon removing device has been described. However, it is conceivable that balloons have to be retrieved to which no catheter shaft is connected. Hence, the pull can not to be exerted by use of a shaft. For this case, the balloon removing device may comprise a gripping element 115 as it is also shown in FIGS. 8 and 9. In the embodiment shown in this figures the gripping element 115 comprises a rod-like member 117 with a distal end at which a hook 119 is located. By means of the hook 119, a balloon from which no catheter shaft 19 or a too short catheter shaft extends can be retrieved. To accomplish this, the gripping element is moved through the lumen 106 of the tube 101 and the wall of the balloon is pierced by the hook 119 so as to introduce the hook into the balloon. When, after piercing the balloon, the gripping element 115 is pulled back through the lumen 106 the hook fixes the balloon at the gripping element so that the balloon can be pulled into or through the lumen 106 of the tube 101 by pulling at the rod-like member 117. Piercing the balloon with the hook 119 can also be used for deflating the balloon if it is not already deflated.

An alternative design of the gripping element 115 is shown in FIG. 11. In this embodiment, the rod-like member is formed by a hollow shaft 217 with a distal end at which a claw-like structure 219 is located. The claw-like structure is formed from a first and a second structure element 219 having free arcuate and acute distal ends 220a, 220b, and proximal ends which are joined at a hinge 218. In addition, a bar which is guided through the interior of the hollow shaft 217 is also connected to the hinge 218. A spring 223 or any other resilient element forces the distal end 220a, 220b of the claw-like structure 219 away from each other.

The claw-like structure 219 can at least partly be retracted into the hollow shaft 217 by means of the bar 221. When the claw-like structure 219 is retracted into the hollow shaft 217 the arcuate distal ends 220a, 220b are moved together against the spring force of the spring 223. This movement can be used for gripping the membrane of the balloon and holding the balloon with the claw-like structure 219. In addition to holding the balloon, the acute distal ends 220a, 220b of the claw-like structure can be used to pierce the balloon for deflating it if it is not already deflated. With the balloon thus fixed at the gripping element 215 the balloon can be pulled into or through the lumen of the tube by pulling at the shaft of the gripping element 215.

Although the gripping elements have been described with respect to the second embodiment and its modification a gripping element can as well be used together with the balloon removing hemostat described with respect to FIGS. 1 to 3 or with the modification described with respect to FIG. 7.

The inventive balloon catheter removing device as has been described by way of example with reference to the embodiments shown in the figures allows for removing a deflated balloon from a body cavity without stretching the access opening to the body cavity, hence avoiding unnecessary pain for the patient.

What is claimed is:

1. A balloon removing device for removing a balloon from a body cavity through an access opening of said body cavity, in particular for removing a balloon of a radiation catheter from a body cavity left by surgical removal of cancerous or other tissue, comprising:
an instretchable elongated tool extending along a longitudinal axis and having a distal end with a distal opening, a proximal end with a proximal opening and a lumen extending from the distal opening along the longitudinal axis to the proximal opening, wherein
the dimensions of said distal opening and said lumen of the elongated tool are adapted to allow the balloon to be accommodated therein in the deflated state, or the dimensions of said distal opening, said proximal opening and said lumen of the elongated tool are adapted to allow the balloon to be pulled through the lumen in the deflated state.

2. The balloon removing device as claimed in claim 1 in which the elongated tool has an inner surface surrounding the lumen and having a low friction.

3. The balloon removing device as claimed in claim 2 in which the inner surface is formed by a low friction coating applied onto the material of the elongated tool.

4. The balloon removing device as claimed in claim 3 in which the low friction coating is a polytetrafluoroethylene coating.

5. The balloon removing device according to claim 2, in which the elongated tool is made of a material with low friction.

6. The balloon removing device as claimed in claim 1 in which the distal opening has a larger cross section than the lumen and in which a tapering wall section extends from the distal opening in axial direction the tapering wall section having a cross section which scales down from the cross section of the distal opening to the cross section of the lumen.

7. The balloon removing device as claimed in claim 1 in which the elongated tool is a tube.

8. The balloon removing device as claimed in claim 1 in which
the elongated tool is formed from two separate pieces which are movable relative to each other in a direction perpendicular to the extension direction of the longitudinal axis of the elongated tool,
a handling element is present which comprises two handling pieces extending substantially perpendicular to the longitudinal axis of the elongated tool wherein each piece of the elongated tool is attached or attachable to a different one of the handling pieces and wherein the handling pieces are movably connected to each other so as to allow to impart a movement to attached pieces of the elongated tool in a direction perpendicular to the longitudinal axis of the elongated tool by moving the handling pieces relative to each other.

9. The balloon removing device as claimed in claim 8 in which the handling element is a tweezers-like, a pliers-like or a scissors-like element wherein the handling pieces are in form of levers each having a manipulation section or end for manipulating the handling element and an attaching section or end to which one of said pieces of the elongated tool is attached or attachable and wherein the levers are pivotally joined together.

10. The balloon removing device as claimed in claim 8 in which the handling element is a pliers-like or a scissors-like element wherein the levers are pivotally joined together.

11. The balloon removing device as claimed in claim 1 in which the elongated tool comprises a holding element for preventing the balloon from slipping out of the lumen of the elongated member through the distal opening.

12. The balloon removing device as claimed in claim 11, in which the holding element comprises one or more resilient tongues projecting from a wall of the elongated tool towards the proximal opening into the lumen.

13. The balloon removing device as claimed in claim 1 which comprises a gripping element for gripping the balloon which can be moved back and forth through the lumen of said elongated tool.

14. The balloon removing device as claimed in claim 13 in which the gripping element has a hook-like or claw-like structure at its distal end.

15. Balloon removing hemostat having a first lever, a second lever and a tube comprising a first semi-tubular piece and a second semi-tubular piece which are made of an instretchable material and which can abut each other so as to form said tube, wherein each lever has a grip end for handling the hemostat and a tool end to which one of the semi-tubular pieces is attached or attachable and wherein the levers are pivotally joined at a location between their grip ends and their tool ends so as to allow a back and forth movement of the semi-tubular pieces from a position where the semi-tubular pieces are separate from each other into a position where the semi-tubular pieces abut each other by handling the grip ends.

16. The balloon removing hemostat as claimed in claim 15 which comprises a rod having a proximal end, a distal end and a hook or claw located at its distal end, the rod being slidably inserted or insertable into the lumen of the tube so that the hook or claw can be moved out of the distal end of the tube to fasten the balloon to the distal end of the rod by the hook or claw and back into or through the tube when the balloon is fastened to the hook or claw.

17. A balloon removing tube being insertable into a body cavity through an access opening of said body cavity, tube being made of an instretchable material and having an axial direction, a distal end with a distal opening, a proximal end with a proximal opening and a lumen extending from the distal opening in axial direction of the tube to the proximal opening, the dimensions of said distal opening and said lumen being adapted to allow the balloon catheter to be accommodated in the lumen in the deflated state or the dimensions of said distal opening, said proximal opening and said lumen being adapted to allow the balloon catheter to be pulled through the lumen in the deflated state.

18. The balloon removing tube as claimed in claim 17 which comprises a rod having a proximal end, a distal end, and a hook or claw located at the distal end of the rod, the rod being slidably inserted or insertable into the lumen of the tube so that the hook or claw can be moved out of the distal end of the tube to fasten the balloon to the distal end of the rod by the hook or claw and back into the tube when the balloon is fastened to the hook or claw.

19. A method of removing a balloon from a body cavity through an access opening of said body cavity, in particular of removing a balloon of a radiation catheter from a body cavity left by surgical removal of cancerous or other tissue, comprising the steps of:
inserting an instretchable elongated tool extending along a longitudinal axis and having a distal end with a distal opening, a proximal end with a proximal opening and a lumen extending from the distal opening to the proximal opening, through the access opening into the body cavity,
pulling the balloon into the lumen of the elongated tool and retrieving the balloon placed in the lumen together with the elongated tool from the body cavity, or retrieving the balloon from the body cavity through the lumen of the elongated tool.

20. The method of claim 19, in which further comprises the step of gripping the balloon with a gripping element which can be moved back and forth through the lumen of said elongated tool, and retrieving the balloon from the body cavity by pulling the balloon into or through the lumen of the elongated tool by means of the gripping element.
